(19) United States
(12) Patent Application Publication
(10) Pub. No.: US 2007/0059200 A1
(43) Pub. Date: Mar. 15, 2007

(54) METHOD OF SANITATION USING CARBON DIOXIDE BASED PROCESS

(76) Inventors: James T.C. Yuan, Naperville, IL (US); Henry J. Ledon, Versailles (FR); John S. Novak, Naperville, IL (US)

Correspondence Address: Ms. Linda K. Russell, Air Liquide Intellectual Property Dept., 2700 Post Oak Blvd., Suite 1800, Houston, TX, 77056, US

(21) Appl. No.: 11/457,593

(22) Filed: Jul. 14, 2006

Related U.S. Application Data
(60) Provisional application No. 60/716,010, filed on Sep. 9, 2005.

(51) Int. Cl. A61L 2/18

(52) U.S. Cl. 422/28; 422/37; 422/34; 422/33

(57) ABSTRACT

Methods and apparatuses are provided for sanitizing food contact surfaces and food processing equipment using a combination of carbon dioxide (CO₂) and antimicrobial chemicals. Generally, the invention provides a method for antimicrobial sanitation, comprising placing an object in a vessel containing an antimicrobial chemical, providing CO₂ into the vessel, and removing the object from the vessel after a given treatment time during which the object is exposed to the provided CO₂ and the antimicrobial chemical.

### CROSS-REFERENCE TO RELATED APPLICATIONS

This application claims the benefit under 35 U.S.C. § 119(e) to provisional application No. 60/716,010, filed Sep. 9, 2005, the entire contents of which are incorporated herein by reference.

### BACKGROUND

1. Field of the Invention

The invention relates to the improvement of sanitization techniques used during the processing of food products, more specifically to a method of sanitizing food contact surfaces and food processing equipment using a combination of carbon dioxide (CO₂) and antimicrobial chemicals.

2. Description of the Related Art

Food safety problems not only originate in the food product itself (e.g., raw ingredients), but also from the environment surrounding the food product. A food product is susceptible to microbial contamination during the processing steps and after the terminal heating process. Operations with poor sanitation in the packing environment can significantly increase the risk of contaminating a food product. For example, pathogenic microorganisms may be found on the floors and in the drains in the packing facility and on the surfaces of sorting, grading, and packing equipment. Without good sanitary practices, any of these surfaces that come in contact with a food product could be a potential source of microbial contamination.

According to at least one estimate, post/cross contamination from either environment or food contact surfaces is implicated in up to 30% of food poisoning cases. Post/cross contamination also increases the microbial load in finished products, shortening shelf-life and becoming a visual deterrent of quality. As such, the finished product can serve as a carrier of cross-contamination leading to economic losses, as well as health and survival issues involving consumers. For example, Listeria spp is an environmental air-borne pathogen causing listeriosis that can contaminate food products during processing. According to the Center for Disease Control, there were 1850 cases of listeriosis in 1998, including 435 deaths from this disease. Effective methods using sanitizers/disinfectants are crucial to minimize and prevent microbial contamination of foods.

Currently, food processing operations use heat, radiation, or antimicrobial chemicals to perform the sanitation process. Thermal sanitization involves the use of hot water or steam for a specified temperature and contact time. Heat (usually above 140° F.) is the most popular method used to clean the floor, walls, and food contact surfaces. Unfortunately, the efficiency of thermal sanitization is low on large exposed surface areas. Chemical sanitizers include chlorine based and quaternary ammonium compounds. They are relatively effective against microorganisms and are inexpensive. However, each type of chemical sanitizer has a limited spectrum of activity and has inherent problems associated with toxicity, shelf-life, and altered tastes. For example, chlorine is effective at pH 6-8, and becomes less effective outside that pH range. Fogging chemicals are anti-microbial compounds in gaseous form that can be used to sanitize pipes and ducts. Fogging chemicals are effective on horizontal food contact surfaces, but not on vertical layouts. Food irradiation is the process of exposing food to ionizing radiation in order to sterilize or preserve food products. Under certain circumstances some research suggests that irradiation forms new chemicals in food, some of which are may be harmful. Irradiation can also reduce the amount of vitamins and other essential nutrients in food products, in addition to negatively impacting the flavor, odor and texture.

Many sanitization methods have been investigated for use in the processing of food products. However, most of them are either ineffective on certain microorganisms or dangerous to the consumers or environment.

Therefore, there remains a need for an innovative process to minimize and/or eliminate microbial contamination in food products caused by contact with various surfaces, food processing equipment, and the environment.

### SUMMARY

Aspects of the invention generally provide a method of sanitizing food contact surfaces and food processing equipment using a combination of CO₂ and antimicrobial chemicals. In one embodiment, the invention provides a method for antimicrobial sanitation, comprising placing an object in a vessel containing an antimicrobial chemical, providing CO₂ into the vessel, and removing the object from the vessel after a given treatment time during which the object is exposed to the provided CO₂ and the antimicrobial chemical.

In another embodiment, the invention provides a method for antimicrobial sanitation, comprising placing an object in a vessel containing an antimicrobial chemical, providing CO₂ into the vessel, providing one or more inert gases into the vessel, and removing the object from the vessel after a given treatment time during which the object is exposed to the provided CO₂, the one or more inert gases, and the antimicrobial chemical.

In another embodiment, the invention provides a method for antimicrobial sanitation, comprising placing an object in a vessel, providing a mixture of CO₂ and one or more antimicrobial chemicals into the vessel, and removing the object from the vessel after a given treatment time during which the object is exposed to the provided mixture.

In another embodiment, the invention provides a method for antimicrobial sanitation, comprising placing an object in a vessel, providing a mixture of CO₂, one or more antimicrobial chemicals, and one or more inert gases into the vessel, and removing the object from the vessel after a given treatment time during which the object is exposed to the provided mixture.

In another embodiment, the invention provides an apparatus for antimicrobial sanitation, comprising a vessel for receiving an object to be sanitized, a CO₂ source in fluid communication with the vessel, and a fluid distribution device inside the vessel in fluid communication with the CO₂ source.

In another embodiment, the invention provides an apparatus for antimicrobial sanitation, comprising a vessel for receiving an object to be sanitized, a CO₂ source in fluid communication with the vessel, one or more inert gas sources in fluid communication with the vessel, and a fluid distribution device inside the vessel in fluid communication with the CO₂ source and the one or more inert gas sources.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 exhibits the main processing steps entailed by the embodiments of the invention.

FIG. 2 shows the apparatus according to one embodiment of the invention.

FIG. 3 shows the combined antimicrobial effect of CO₂ and alcohol (ROH) according to one preferred embodiment of the invention.

### DESCRIPTION OF PREFERRED EMBODIMENTS

The words and phrases used herein should be given their ordinary and customary meaning in the art by one skilled in the art unless otherwise further defined.

In the following, reference is made to embodiments of the invention. However, it should be understood that the invention is not limited to specific described embodiments. Instead, any combination of the following features and elements, whether related to different embodiments or not, is contemplated to implement and practice the invention. Furthermore, in various embodiments the invention provides numerous advantages over the prior art. However, although embodiments of the invention may achieve advantages over other possible solutions and/or over the prior art, whether or not a particular advantage is achieved by a given embodiment is not limiting of the invention. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

A food product is susceptible to microbial contamination during virtually all steps of preparation. Aspects of the invention generally provide a method of sanitizing food contact surfaces and food processing equipment using a combination of CO₂ and antimicrobial chemicals to eliminate or significantly reduce microbial contamination.

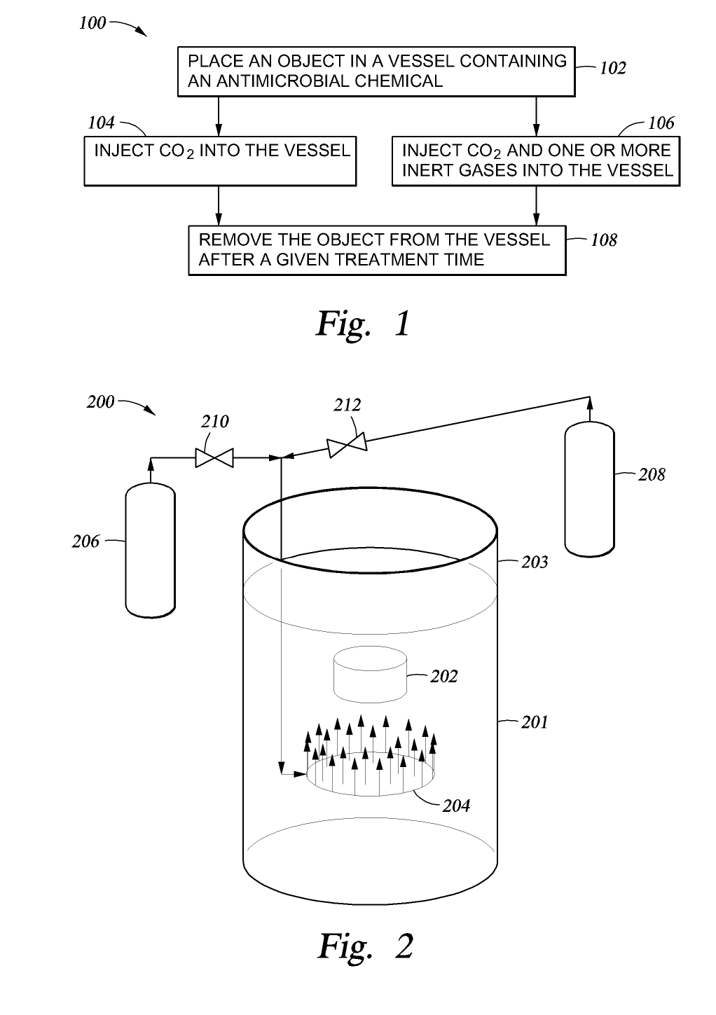

FIG. 1 is a flow diagram of a process 100, according to one embodiment of the present invention. The process 100 includes a processing step 102 involving a placement of an object in a vessel containing an antimicrobial chemical and a processing step 104 involving an injection of CO₂ into the vessel. Another embodiment of the invention includes an alternative processing step 106 involving an injection of CO₂ and one or more inert gases into the vessel. The process 100 further includes a processing step 108 involving a removing the object from the vessel after a given treatment time.

The processing steps 102-108 according to the embodiments of the invention are described below. The embodiments described herein are provided to illustrate the invention and the particular embodiments shown should not be used to limit the scope of the invention.

The first processing step 102 of the invention involves placement of an object in a vessel containing an antimicrobial chemical. Any type of vessel may be employed in the invention. A particular embodiment involves the use of a beaker as the vessel. The object can remain submerged in the vessel by any type of support. In one embodiment, a carousel in the vessel contains slots which enable food contact surfaces to be inserted without touching the sides of the slots, allowing the antimicrobial fluid in the vessel to treat all surfaces. The carousel is held by a screen that is above a sparger. Antimicrobial chemicals that can be used in embodiments of processing step 102 include any chemical that can kill microorganisms. Examples include hydrogen peroxide (H₂O₂), ethylene oxide, and chlorine. One embodiment of this invention employs a vessel filled with sterilized water containing about 0% to about 13% ethanol, an antimicrobial alcohol.

The next processing step 104 according to one embodiment of the invention involves providing CO₂ in the vessel from a gaseous CO₂ source. The CO₂ source is connected to a porous sparger located beneath the object submerged in processing step 102. Porous spargers allow gaseous CO₂ to enter the vessel in the form of bubbles and diffuse throughout the solution containing the antimicrobial chemical chosen in processing step 102. In one embodiment, CO₂ gas at room temperature is sparged through the solution in the vessel at a rate of 5 grams per minute. In this embodiment, the pressure of the vessel may be maintained at 10 psi. Gaseous CO₂ can be sparged through the solution for an appropriate time according to the size and features of the object. Some embodiments of this invention involve injecting CO₂ into the vessel for a time period up to 40 minutes. In another embodiment, gaseous CO₂ and an antimicrobial chemical can be mixed to create a carbonated fluid. This carbonated fluid can then be introduced and applied to an object located in a vessel for a given treatment time.

One or more inert gases may be injected into the vessel from a source in processing step 106 to help tune and maintain the partial pressure and alter properties such as the concentration of the injected gaseous CO₂. The one or more inert gases can be pre-mixed with CO₂ in a separate vessel and injected together, or injected separately. The inert gases that can be used include, for example, H₂, O₂, NO, N₂O, N₂, He, Ar, Kr, Xe, and various combinations and ratios thereof. Processing step 108 involves removing the object from the vessel in a sterile manner after a given treatment time. In particular embodiments, sterile tongs or tweezers are used to remove the objects from the vessel in processing step 108. The addition or removal of objects from the vessel could be automated or robotized in further embodiments.

FIG. 2 shows the apparatus 200 according to one embodiment of the invention. An object 202 is immersed and suspended in a vessel 203 containing a solution 201. The solution 201 consists of a specific antimicrobial formulation. A gaseous CO₂ source 206 is connected to a sparger 204 through which CO₂ bubbles are able to enter the solution in the vessel by passing through a valve 210. An inert gas source 208 is also connected to the sparger in this embodiment by means of a valve 212. In a particular embodiment, the sparging flow from the sparger 204 can be controlled with a gas flow regulator and rotameter to avoid over-foaming or bubbles in the vessel 203. The inert gas can be premixed with CO₂ before entering the vessel, or injected separately. One embodiment can employ an automated gas mixer (PBI Dansensor, Glen Rock, N.J.) to mix one or more inert gases and CO₂ from separate cylinders in a vessel or storage container. The gas mixture can then be verified in this embodiment using a food package analyzer (such as one available from Servomex, Inc., of Sugar Land, Tex.) or gas chromatograph. The gas mixture can be contained in a buffer vessel and then released to the antimicrobial solution.

### EXAMPLE

In another embodiment, the process 100 was conducted to demonstrate the combined antimicrobial effect of CO₂ and different concentrations of ethanol (with approximately 8% methanol and isopropanol) on the reduction of Escherichia coli JM109 contamination on stainless steel coupons (1 inch×2 inch). The stainless steel coupons were washed with soap and water, rinsed, dried, and sterilized by autoclaving. One side of the steel coupons contained an etched number that enabled distinction of inoculated versus untreated surfaces. Cultures of E. Coli JM109 were grown in tryptic soy broth (TSB) overnight at 37° C. One hundred microliters of the E. Coli culture were spotted in small increments over the unetched side of the steel coupon and allowed to dry in a laminar flow hood at room temperature for 30 minutes. The inoculated steel coupons were then placed in a coupon separator and submerged in a beaker filled with 0 to 13% alcohol. CO₂ gas at room temperature was sparged through the solution at a rate of 5 grams per minute, and the beaker was maintained at a pressure of 10 psi. Every 5 minutes from 0 to 40 minutes, a sample coupon was removed from the coupon separator and the inoculated surface was swabbed 3 times with a sterile cotton swab. The cotton swab was diluted and washed vigorously in plating medium in a labeled sterile test tube. Vortexed sample tubes were serially diluted in sterile peptone-water and plated on brain-heart infusion agar (BHIA) plates. Following incubation at 37° C. overnight, BHIA plates were counted for colony forming units (CFU) per ml and results recorded.

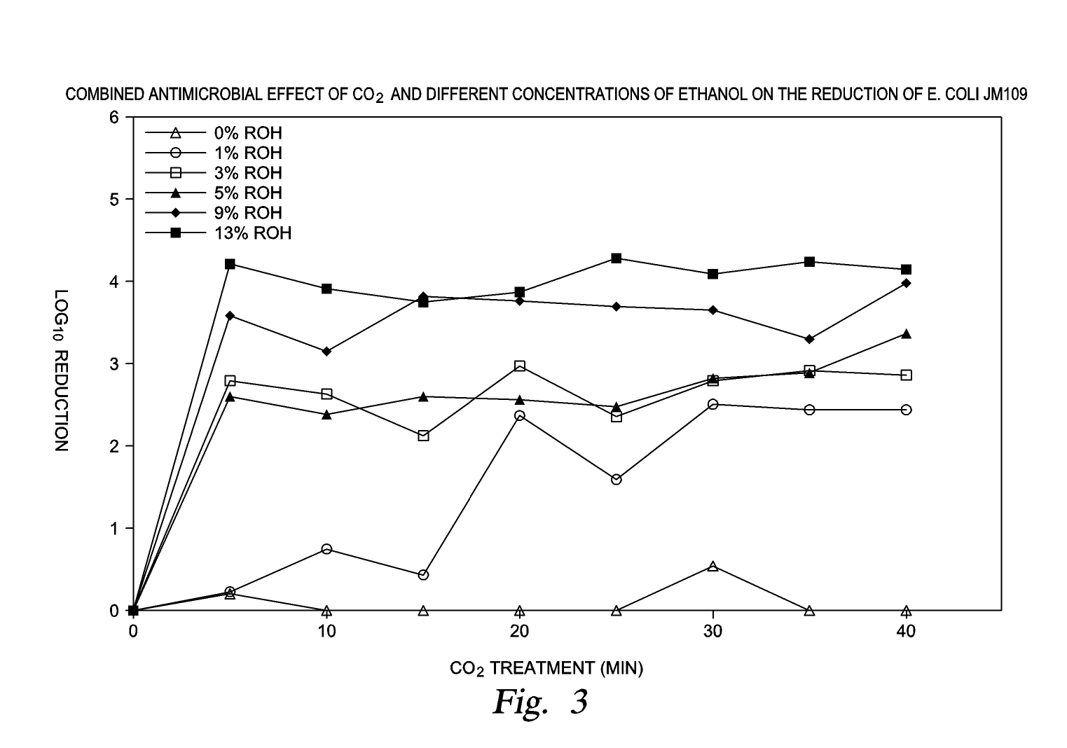

FIG. 3 exhibits the combined antimicrobial effect of CO₂ and different concentrations of ethanol on the reduction of Escherichia coli JM109 contamination on the stainless steel coupons. The greatest reduction in E. coli contamination occurred when the stainless steel coupons were exposed to CO₂ and 13% ROH. Virtually no antimicrobial effects were seen in steel coupons exposed to 0% ROH and up to 40 minutes of CO₂ treatment. The antimicrobial effectiveness of CO₂ combined with ROH is also exemplified by the following: exposing the steel coupons to 0% ROH and 20 minutes of CO₂ exposure resulted in no (0%) reduction in E. coli survival, but 1% ROH and 20 minutes of CO₂ exposure resulted in a 2 log 10 (99%) reduction in E. coli survival. Furthermore, 1% ROH and 20 minutes of CO₂ exposure led to a 0.5 log 10 (31.2%) reduction in E. coli survival, while 1% ROH and 40 minutes of CO₂ exposure led to a 2.5 log 10 (99.7%) reduction in E. coli survival. The results shown in FIG. 3 demonstrate that a combination of CO₂ and antimicrobial chemicals can be used to sanitize objects involved in food processing, and improve microbial safety of finished food products.

Preferred processes and apparatus for practicing the present invention have been described. It will be understood and readily apparent to the skilled artisan that many changes and modifications may be made to the above-described embodiments without departing from the spirit and the scope of the present invention. The foregoing is illustrative only and that other embodiments of the integrated processes and apparatus may be employed without departing from the true scope of the invention defined in the following claims.

What is claimed is:

1. A method for antimicrobial sanitation, comprising:
a) placing an object in a vessel containing an antimicrobial chemical;
b) providing carbon dioxide (CO₂) into the vessel; and
c) removing the object from the vessel after a given treatment time during which the object is exposed to the CO₂ and the antimicrobial chemical.

2. The method of claim 1, wherein the antimicrobial chemical is selected from the group consisting of alcohols, H₂O₂, ethylene oxide, chlorine, and combinations thereof.

3. The method of claim 2, wherein the alcohol is selected from the group consisting of isopropanol, methanol, butanol, ethanol, and combinations thereof.

4. The method of claim 1, wherein the pressure in the vessel is maintained at between 14 psi and 15,000 psi.

5. The method of claim 1, wherein the temperature in the vessel is maintained at between 0° C. and 100° C.

6. A method for antimicrobial sanitation, comprising:
a) placing an object in a vessel containing an antimicrobial chemical;
b) providing CO₂ into the vessel;
c) providing one or more inert gases into the vessel; and
d) removing the object from the vessel after a given treatment time during which the object is exposed to the CO₂, the one or more inert gases, and the antimicrobial chemical.

7. The method of claim 6, wherein the antimicrobial chemical is selected from the group consisting of alcohol, H₂O₂, ethylene oxide, chlorine, and combinations thereof.

8. The method of claim 7, wherein the alcohol is selected from the group consisting of isopropanol, methanol, butanol, ethanol, and combinations thereof.

9. The method of claim 6, wherein the inert gas is selected from the group consisting of H₂, O₂, NO, N₂O, N₂, He, Ar, Kr, Xe, and combinations thereof.

10. The method of claim 6, wherein the pressure in the vessel is maintained at between 14 psi and 15,000 psi.

11. The method of claim 6, wherein the temperature in the vessel is maintained at between 0° C. and 100° C.

12. A method for antimicrobial sanitation, comprising:
a) placing an object in a vessel;
b) providing a mixture of CO₂ and one or more antimicrobial chemicals into the vessel; and
c) removing the object from the vessel after a given treatment time during which the object is exposed to the mixture.

13. The method of claim 12, wherein the antimicrobial chemical is selected from the group consisting of alcohol, H₂O₂, ethylene oxide, chlorine, and combinations thereof.

14. The method of claim 13, wherein the alcohol is selected from the group consisting of isopropanol, methanol, butanol, ethanol, and combinations thereof.

15. The method of claim 12, wherein the pressure in the vessel is maintained at between 14 psi and 15,000 psi.

16. The method of claim 12, wherein the temperature in the vessel is maintained at between 0° C. and 100° C.

17. A method for antimicrobial sanitation, comprising:
a) placing an object in a vessel;
b) providing a mixture of CO₂, one or more antimicrobial chemicals, and one or more inert gases into the vessel; and
c) removing the object from the vessel after a given treatment time during which the object is exposed to the provided mixture.

18. The method of claim 17, wherein the antimicrobial chemical is selected from the group consisting of alcohol, H₂O₂, ethylene oxide, chlorine, and combinations thereof.

19. The method of claim 18, wherein the alcohol is selected from the group consisting of isopropanol, methanol, butanol, ethanol, and combinations thereof.

20. The method of claim 17, wherein the inert gas is selected from the group consisting of H₂, O₂, NO, N₂O, N₂, He, Ar, Kr, Xe, and combinations thereof.

21. The method of claim 17, wherein the pressure in the vessel is maintained at between 14 psi and 15,000 psi.

22. The method of claim 17, wherein the temperature in the vessel is maintained at between 0° C. and 100° C.

23. An apparatus for antimicrobial sanitation, comprising:
a) a vessel for receiving an object to be sanitized;
b) a CO₂ source in fluid communication with the vessel;
c) a fluid distribution device inside the vessel in fluid communication with the CO₂ source; and
d) valving for controlling the flow of CO₂ from the CO₂ source to the fluid distribution device.

24. The apparatus of claim 23, wherein the fluid distribution device is a sparger.

25. An apparatus for antimicrobial sanitation, comprising:
a) a vessel for receiving an object to be sanitized;
b) a CO₂ source in fluid communication with the vessel;
c) one or more inert gas sources in fluid communication with the vessel; and
c) a fluid distribution device inside the vessel in fluid communication with the CO₂ source and the one or more inert gas sources.

26. The apparatus of claim 25, wherein the fluid distribution device is a sparger.
